(19) United States
(12) Patent Application Publication
(10) Pub. No.: US 2022/0096512 A1
(43) Pub. Date: Mar. 31, 2022

(54) WATER-BASED ANTIFUNGAL COMPOSITIONS

(71) Applicant: Sigma-Aldrich Co. LLC, St. Louis, MO (US)

(72) Inventors: Aviran Amir, Rosh Haayn (IL); Tamar Dvash, Kibbutz Tzuba (IL); Michal Levin-Khalifa, Jerusalem (IL)

(21) Appl. No.: 17/426,946

(22) PCT Filed: Jan. 31, 2020
(86) PCT No.: PCT/US2020/016107

§ 371 Date: Jul. 29, 2021

Related U.S. Application Data
(60) Provisional application No. 62/799,434, filed on Jan. 31, 2019.

(51) Int. Cl. A61K 31/7048; A61K 47/18; A61K 9/19

(52) U.S. Cl. CPC A61K 31/7048; A61K 9/19; A61K 47/18

(57) ABSTRACT

Water-based antifungal composition having improved bioavailability; such formulations include a polyene macrolide antifungal agent, such as nystatin, a surfactant, a buffer, an organic amine base; and water. Also provided are methods for preparing water-based antifungal compositions.

### CROSS-REFERENCE TO RELATED APPLICATIONS

This application claims the benefit of prior to U.S. Provisional Patent Application No. 62/799,434, filed Jan. 31, 2019, the entirety of which is incorporated herein by reference.

### BACKGROUND

Various polyene macrolide compounds found in nature, such as nystatin, amphotericin, candicidin, natamycin, polyfungin, and others have antifungal properties. These polyene macrolide compounds have found use as antifungal agents in various applications, including, for example, biocides in cell culture, and treating fungal infections in humans and animals. Nystatin, for example is an anti-fungal small molecule commonly used in clinic and for research.

Due to the polyene nature of these compounds, polyene macrolide antifungal agents are water insoluble, therefore the bioavailability of these compounds in aqueous based systems is reduced. This generates a major challenge to introduce these antifungal agents, for instance, to live cell culture where media is an aqueous solution. Currently available nystatins are able to either solubilize in DMSO/DMF or remain in suspension in aqueous media.

Moreover, the insolubility in water impacts the activity of these compounds and may significantly impact the toxicity because high dosages or the use of undesirable solvents may be required for effectiveness. For these reasons, some applications requiring such antifungal agents, particularly clinical applications have not been

A need exists for new antifungal compositions having improved properties, and thus improved bioavailability, for both research and clinical applications.

### SUMMARY

Provided herein are water-based antifungal composition having improved bioavailability; such formulations include a polyene macrolide antifungal agent, a surfactant, a buffer, an organic amine base; and water.

In various embodiments, the water-based antifungal compositions are selected from nystatin, amphotericin, candicidin, natamycin, polyfungin, and Levorin. In some embodiments, the water-based antifungal compositions described herein can have a combination of antifungal agents. In a preferred embodiment, the polyene macrolide antifungal agent is nystatin.

In various embodiments, the surfactant is selected from the group consisting of 3-[(3-cholamidopropyl)dimethylammonio]-1-propanesulfonate hydrate (CHAPS), sodium deoxycholate, sodium dodecyl sulfate (SDS), sodium myreth sulfate and combinations thereof. In a preferred embodiment the surfactant is sodium deoxycholoate. The surfactant concentration may range from about 1 to about 8 equivalents of surfactant per equivalent of polyene macrolide antifungal. In a preferred embodiment, the surfactant is present in the range from 2-4 eq per eq of polyene macrolide antifungal.

The buffer used in the water-based antifungal is preferably a biological buffer. In some embodiments, the buffer can include one or more of the following: Na₂HPO₄, TRIS and MOPS. In a preferred embodiment, the buffer includes Na₂HPO₄. The buffered pH of the water-based antifungal composition is preferably in the range from about 5 to about 11. In some embodiments, the pH is in the range from about 6 to about 9. In a preferred embodiment, the pH is in the range from about 7 to about 8. In a particularly preferred embodiment, the pH is in the range from about 7.4 to 7.6.

In various embodiments, the organic amine base may be a quaternary amine base, a tertiary amine base, a secondary amine base, a primary amine base, or a combination of organic amine bases. Some preferred amine bases include propylamine, ethanolamine, diethylamine, dipropylamine, trimethylamine, triethylamine, tripropylamine, diisopropylethylamine, diethyldimethylammonium hydroxide, tetrabutylammonium hydroxide, 4-methylmorpholine, morpholine and piperidine and combinations thereof.

In some embodiments, the composition may further include a salt. In such embodiments, the salt may be Na₂HPO₄, NaCl, KCl, sodium ascorbate, sodium lactate, sodium gallate and combinations thereof.

In some embodiments, the water-based antifungal composition contains at least about 95% water (by weight). In a preferred embodiment, the composition contains at least about 98% water (by weight).

In some embodiments, the composition may be reconstituted from a solid. In these embodiments, the solid may be a powder or a tablet. In some embodiments, the solid is a lyophilized powder.

In a preferred embodiment, the antifungal activity or potency of the composition is at least equivalent to the antifungal activity of the equivalent amount of nystatin solution in DMF as measured by a standard assay method such as agar diffusion assay or cylinder plate assay.

Further described herein are water-based nystatin compositions having improved bioavailability; such compositions include nystatin, 2-4 eq of sodium deoxycholoate per eq of nystatin, triethylamine, a biological buffer, water, and optionally a salt, and have a pH in the range from about 7 to about 8. In preferred embodiments, the buffer is selected from Na₂HPO₄, TRIS, MOPS and combinations thereof. In a particularly preferred embodiment, the buffer includes Na₂HPO₄. In embodiments that include the optional salt, preferred salts include Na₂HPO₄, NaCl, KCl, sodium ascorbate, sodium lactate, sodium gallate and combinations thereof. In a preferred embodiment, the composition has a potency, as measured by agar diffusion assay, that is at least equivalent to the antifungal activity of the equivalent amount of nystatin solution in DMF.

Further provided herein are water-based macrocyclic polyene antifungal compositions containing amphotericin, candicidin, natamycin, polyfungin, Levorin and combinations thereof, but wherein the polyene antifungal agent is not nystatin. Such embodiments also include 2-4 eq of sodium deoxycholoate per eq of polyene antifungal agent, an organic amine, such as triethylamine, a biological buffer, water, and optionally a salt, and have a pH in the range from about 7 to about 8. In preferred embodiments, the buffer is selected from Na₂HPO₄, TRIS, MOPS and combinations thereof. In a particularly preferred embodiment, the buffer includes Na₂HPO₄. In embodiments that include the optional salt, preferred salts include Na₂HPO₄, NaCl, KCl, sodium ascorbate, sodium lactate, sodium gallate and combinations thereof. In a preferred embodiment, the composition has a potency, as measured by agar diffusion assay, that is at least equivalent to the antifungal activity of the equivalent amount of the same macrocyclic antifungal agent in solution in DMF.

Further provided is a method for making a water-based antifungal composition comprising the steps of selecting a polyene macrolide antifungal agent, a biological buffer, and a surfactant; suspending the polyene macrolide antifungal agent, the biological buffer, and the surfactant in water; titrating the suspension with an organic amine base until the suspension becomes a clear solution; and adding an acid to adjust the pH of the solution to the range from about 7 to about 8.

### DETAILED DESCRIPTION

Provided herein are water-based antifungal compositions with higher bioavailability than conventional formulations, such as suspensions or DMSO/DMF based compositions. The compositions provided herein include a polyene macrolide antifungal agent, a surfactant, a buffer, an organic amine base; and water.

### Definitions

As used herein the term “antifungal agent” refers generally to polyene macrolide antifungal agents, sometimes referred to herein simply as polyene macrolide antifungals, antifungals or antifungal agent. Antifungal agents suitable in the compositions and methods described herein include, for example, nystatin, amphotericin, candicidin, natamycin, polyfungin, and Levorin.

The terms “antifungal activity” or “potency,” these terms being used interchangeably, refer to the inhibitory effect of an antifungal agent on microorganisms under suitable conditions as measured using a standard analytical method, such as the methods established by the United States Pharmacopeial Convention (USP).

“Aqueous” or “water-based” as used herein refers to a solution that includes water as the solvent. In various embodiments, the aqueous or water-based compositions described herein may include at least about 90% water by weight, at least about 95% water by weight, at least about 96% water by weight, at least about 97% water by weight, at least about 98% water by weight, or at least about 99% water by weight.

As used herein, the term “bioavailability” refers to the proportion of antifungal agent solubilized in aqueous solution and thus able to have an active effect on microorganisms it comes into contact with.

“Improved bioavailability,” as used herein, means that the bioavailability of antifungal agent is improved when compared with the same amount of antifungal agent in a conventional system, i.e., either suspended in solution, or dissolved in dimethylsulfoxide (DMSO)/dimethylformamide (DMF).

### Composition

The water-based antifungal compositions provided herein have improved bioavailability when tested using standard tests, such as agar diffusion assay, which is described in greater detail below. The compositions provided herein generally include a polyene macrolide antifungal agent, a surfactant, a buffer, an organic amine base; and water. In some embodiments, additional components may be added, such as a salt.

The compositions described herein are illustrated using nystatin as an exemplary polyene macrolide antifungal agent; however, these compositions, and methods used to make these compositions can be adapted for any polyene macrolide antifungal agent or combinations of two or more polyene macrolide antifungal agents. Polyene macrolide antifungal agents useful in the compositions described herein include nystatin, amphotericin, also known as Amphotericin B, candicidin, natamycin, also known as pimaricin, polyfungin, and Levorin, as well as related compounds.

The compositions described herein include a surfactant, such as sodium deoxycholate, 3-[(3-cholamidopropyl)dimethylammonio]-1-propanesulfonate hydrate (CHAPS), sodium dodecyl sulfate (SDS), sodium myreth sulfate and combinations thereof. In a preferred embodiment the surfactant is sodium deoxycholoate.

The surfactant concentration may range from about 1 to about 8 equivalents (eq) of surfactant per equivalent of polyene macrolide antifungal. In one preferred embodiment, the surfactant is present in the range from about 2 to about 4 eq per eq of polyene macrolide antifungal. In other preferred embodiments, the surfactant may be present at a level of 1.5, 2, 2.5, 3, 3.5, 4, 4.5, or 5 eq per eq of polyene macrolide antifungal agent.

The buffer used in the water-based antifungal compositions described herein is selected to maintain the pH of the composition at a pH relevant to the particular application in which the composition will be used. Particularly preferred are “biological buffers,” which are a subset of buffers that are typically organic substances that maintain a constant pH over a given range, by neutralizing the effects of hydrogen ions. The terms “buffer” and “biological buffer” are used interchangeably herein.

Some biological buffers with pKa values in the relevant range for the compositions described herein include phosphate buffer systems, specifically Na₂HPO₄; MOPS (synonyms 3-(N-morpholino)propanesulfonic acid, 4-Morpholinepropanesulfonic acid), TRIS (2-Amino-2-(hydroxymethyl)-1,3-propanediol) available as TRIZMA® base (Sigma-Aldrich, St. Louis, Mo.); PIPES [piperazine-N,N′-bis(2-ethanesulfonic acid)]; HEPES (4-(2-hydroxyethyl)-1-piperazineethanesulfonic acid); and DIPSO (3-(N,N-Bis [2-hydroxyethyl]amino)-2-hydroxypropanesulfonic acid). Combinations of buffer may be used to adjust the buffering capacity over a range of temperatures as well as lower the concentrations of individual buffers used, thus lowering the chance of buffer-related toxicity for particular applications.

In preferred embodiments, the buffer includes one or more of the following: Na₂HPO₄, TRIS and MOPS. In a preferred embodiment, the buffer includes Na₂HPO₄. The buffered pH of the water-based antifungal composition is preferably in the range from about 5 to about 11. In various embodiments, the buffered pH of the composition may be about, for example, 5, 5.5, 6, 6.1, 6.2, 6.3, 6.4, 6.5, 6.6, 6.7, 6.8, 6.9, 7.0, 7.05, 7.1, 7.15, 7.2, 7.25, 7.3, 7.35, 7.4, 7.45, 7.5, 7.55, 7.6, 7.65, 7.7, 7.75, 7.8, 7.85, 7.9, 7.95, 8, 8.1, 8.2, 8.3, 8.4, 8.5, 8.6, 8.7, 8.8, 8.9, 9, 9.0, 9.1, 9.2, 9.3, 9.4, 9.5, 9.6, 9.7, 9.8, 9.9, 10, 10.5, 11. Typically, the target pH will be a range of approximately 1 pH unit, more preferably about 0.5 pH units, even more preferably, about 0.2 pH units, and even more preferably about 0.15 pH units. In some embodiments, the pH is in the range from about 6 to about 9. In a preferred embodiment, the pH is in the range from about 7 to about 8. In a particularly preferred embodiment, the pH is in the range from about 7.4 to 7.55.

The organic amine base is used to aid the dissolution of the polyene macrolide antifungal agent in the compositions described herein. Organic amines were shown to be milder bases than inorganic bases, thus resulting in little to no decomposition of the antifungal agents. The organic amine bases may be quaternary amine bases, tertiary amine bases, secondary amine bases, primary amine bases, cyclic tertiary or secondary amine bases, or a combination of organic amine bases.

Exemplary quaternary amines include but are not limited to diethyldimethylammonium hydroxide and tetrabutylammonium hydroxide.

Exemplary tertiary amines include but are not limited to trimethylamine, triethylamine, tripropylamine, diisopropylethylamine and 4-methylmorpholine.

Exemplary secondary amines include but are not limited to dimethylamine, diethylamine, dipropylamine, piperidine and morpholine.

Exemplary primary amines include but are not limited to methylamine, ethylamine, propylamine, isopropylamine, butylamine, isobutylamine, sec-butylamine, tert-butylamine, ethanolamine, and 2-amino-2-methyl-1-propanol.

Some preferred amine bases include propylamine, ethanolamine, diethylamine, dipropylamine, trimethylamine, triethylamine, tripropylamine, diisopropylethylamine, diethyldimethylammonium hydroxide, tetrabutylammonium hydroxide, 4-methylmorpholine, morpholine and piperidine and combinations thereof

In some embodiments, the composition may further include a salt. The salt is particularly useful in embodiments in which the composition will be dried as a powder, e.g., for storage, shipping and so forth, and then redissolved in water or aqueous solution later. In such embodiments, the salt may be used to obtain a fine powder, which is readily dissolved in water, after lyophilization. In such embodiments, the salt may be, e.g, Na₂HPO₄, NaCl, KCl, sodium ascorbate, sodium lactate, sodium gallate and combinations thereof

In some embodiments, the water-based antifungal composition contains at least about 95% water (by weight). In a preferred embodiment, the composition contains at least about 98% water (by weight).

In some embodiments, the composition may be reconstituted from a solid. In these embodiments, the solid may be a powder or a tablet. In some embodiments, the solid is a lyophilized powder.

In a preferred embodiment, the antifungal activity or potency of the composition is at least equivalent to the antifungal activity of the equivalent amount of nystatin solution in DMF as measured by a standard assay method such as agar diffusion assay or cylinder plate assay.

Further described herein are water-based nystatin compositions having improved bioavailability; such compositions include nystatin, 2-4 eq of sodium deoxycholoate per eq of nystatin, triethylamine, a biological buffer, water, and optionally a salt, and have a pH in the range from about 7 to about 8. In preferred embodiments, the buffer is selected from Na₂HPO₄, TRIS, MOPS and combinations thereof. In a particularly preferred embodiment, the buffer includes Na₂HPO₄. In embodiments that include the optional salt, preferred salts include Na₂HPO₄, NaCl, KCl, sodium ascorbate, sodium lactate, sodium gallate and combinations thereof. In a preferred embodiment, the composition has a potency, as measured by agar diffusion assay, that is at least equivalent to the antifungal activity of the equivalent amount of nystatin solution in DMF.

Further provided is a method for making a water-based antifungal composition comprising the steps of selecting a polyene macrolide antifungal agent, a biological buffer, and a surfactant; suspending the polyene macrolide antifungal agent, the biological buffer, and the surfactant in water; titrating the suspension with an organic amine base until the suspension becomes a clear solution; and adding an acid to adjust the pH of the solution to the range from about 7 to about 8.

Preparation

The compositions described herein may be prepared using the following procedure.

The polyene macrolide antifungal agent, surfactant and buffer are weighed and added to a flask. A suspension is formed by adding water to the flask and vigorously mixing.

The suspension is slowly titrated with the organic amine to pH in the range from about 10.4 to about 10.7, until the solution is clear.

After the solution is clear, acid is slowly added to bring the pH within the desired range, typically about 7 to about 8; about 7.4 to 7.6 in preferred embodiments.

The solution is filtered to a suitable vessel; then the endotoxin level is determined by quantitative testing, e.g., LAL testing. Provided the result is suitable, e.g., ≤0.5 EU/ml at 10 ml/L solution (diluted 1:100 in LAL tested purified water), the potency may then be determined, preferably according to USP methodology. The solution may also be stored at 2-8° C. in the dark.

The final solution may be obtained by dilution to the preferred potency, typically 8000-13,000U/ml.

In some embodiments, formulation may be freeze dried, for ease of storage and shipping, and reconstituted for use when needed.

Potency Testing

Potency testing is done using a cylinder plate assay using the methods described in U.S. Pharmacopeia, e.g., Pharmacopeial Forum, Vol. 36(6) [November-December 2010] <81> Antibiotics—Microbial Assays, USP 32 page 86 ff.

### EXAMPLES

### Example 1

### Preparation of a Nystatin Solution

25 mg Nystatin, 46.6 mg sodium deoxycholate and 2.66 mg Na₂HPO₄ were weighed into a 4 L Erlenmeyer flask. The powders were suspended in 2.3 L distilled deionized (dd) water and mixed vigorously using an overhead stirrer. The solution was titrated slowly with Et3N to pH 10.7. After the solution became clear, about 0.1N HCl was added to bring the pH within the range of 7.4-7.55. The solution was filtered to a suitable vessel.

The endotoxin level was tested by LAL test; and confirmed to be ≤0.5 EU/ml at 10 ml/L solution (diluted 1:100 in LAL tested PW). The potency of the solution was determined according to method USP/81. The unused solution was stored at 2-8° C. in the dark

After determination of the potency, the solution was diluted to the required potency with purified water.

### Comparative Example

Formulations that did not work included using strong bases such as NaOH and KOH in order to bring the pH to 11-12:

2.2 g of Na₂HPO₄ and 8.23 g of NaCl were dissolved in 3 L of dd water into 5 L container. Assure total solubilization.13.17 g of deoxycholic acid-sodium salt was carefully added and it is totally dissolved. 26.3 g of Nystatin was added. then the mixture was stirred vigorously until all powder suspended and a homogeneous solution was obtained.

The mixture was titrated to approximately pH 11.8 with 5N NaOH, and stirred. The pH was then lowered to approximately 7.5 with 5N HCl The resulting solution showed an unacceptable level of decomposition of the nystatin.

Minimum inhibitory concentration (MIC) testing was determined for the inventive nystatin formulation described herein, along with comparative antifungal agents.

Nystatin (Solid) and amphotericin were dissolved in DMF in 1 mg/mL. Econazole nitrate was dissolved in PBS to achieve 1 mg/mL. Nystatin formulation diluted with PBS to achieve the same potency as Nystatin (Solid).

Antibiotic medium 19 was sterilized in autoclave, after sterilization when the temperature lowered to 45° C. and 1 mL from spore suspension was added. 8 mL from this mixture dispensed to each petri dish.

On each dish 6 sterile diffusion paper disc were placed, 10 μL from each antibiotic were applied in 10, 15, 20, 25, 30, 35 μg/mL concentrations. Where MIC concentrations were below 10, lower concentrations were used: 2, 5, 7.5 μg/mL

The petri dishes were incubated overnight at 30° C. The MIC were determined as the lower concentration that inhibition could be visualized. The results are summarized in Table 1, below.

TABLE 1
Inhibition of Candida albicans and Aspergillus niger
in μg/mL, each result is an average of three experiments.

| Antifungal Agent | Candida albicans | Aspergillus niger |
|---|---|---|
| Amphotericin | 16 | 31.5 |
| Econazole | No inhibition | No inhibition |
| Nystatin formulation** | 6.7 | 12.5 |
| Nystatin | 14 | 22.5 |
| Polymixin B | No inhibition | No inhibition |
| Nystatin Methyl ester | 30 | No inhibition |

Observations. In this diffusion disc assay we measured the minimal concentration applied by each compound needed to inhibit the growth of C. albicans and A. niger. Nysatin inhibited the growth of C. albicans and A. niger at 14 μg/mL and 22.5 μg/mL, respectively. However, Nystatin formulation was more active in inhibiting the growth of C. albicans and A. niger (6.7 and 12.5 μg/mL respectively). Interestingly, Nystatin methyl ester was found to be less active (30 μg/mL) against C. albicans and with no observed inhibition for A. niger. Amphotericin B inhibit the growth of C. albicans (16 μg/mL) similarly to Nystatin. However, Amphotericin B was less active (31.5 μg/mL) in inhibiting the growth of A.niger.

The examples provided herein are for illustrative purposes only and are not meant to limit the scope of the invention as defined by the claims.

1. A water-based antifungal composition having improved bioavailability comprising:
a polyene macrolide antifungal agent selected from the group consisting of nystatin, amphotericin, candicidin, natamycin, polyfungin, Levorin and combinations thereof,
a surfactant selected from the group consisting of 3-[(3-cholamidopropyl)dimethylammonio]-1-propanesulfonate hydrate (CHAPS), sodium deoxycholate, sodium dodecyl sulfate (SDS), sodium myreth sulfate and combinations thereof,
a biological buffer selected from the group consisting of Na₂HPO₄, TRIS, MOPS and combinations thereof,
an organic amine base; and
water.

2. (canceled)

3. The composition of claim 1 wherein the polyene macrolide antifungal agent is nystatin.

4. (canceled)

5. (canceled)

6. (canceled)

7. The composition of 1 wherein the buffer comprises Na₂HPO₄.

8. The composition of claim 1 wherein the surfactant is present in the range from 1-8 equivalents (eq) per eq of polyene macrolide antifungal.

9. The composition of claim 8 wherein the surfactant is present in the range from 2-4 eq per eq of polyene macrolide antifungal.

10. The composition of claim 9 wherein the surfactant is sodium deoxycholate.

11. The composition of claim 1 having a pH in the range from about 5 to about 11.

12. The composition of claim 11 wherein the pH is in the range from about 6 to about 9.

13. The composition of claim 12 wherein the pH is in the range from about 7 to about 8.

14. The composition of claim 13 wherein the pH is in the range from about 7.4 to 7.6.

15. The composition of claim 1 wherein the organic base is selected from the group consisting of quaternary amine bases, tertiary amine bases, secondary amine bases, primary amine bases and combinations thereof.

16. The composition of claim 15 wherein the organic base is selected from the group consisting of propylamine, ethanolamine, diethylamine, dipropylamine, trimethylamine, triethylamine, tripropylamine, diisopropylethylamine, diethyldimethylammonium hydroxide, tetrabutylammonium hydroxide, 4-methylmorpholine, morpholine and piperidine and combinations thereof.

17. The composition of claim 1 further comprising a salt.

18. The composition of claim 17 wherein the salt is selected from the group consisting of Na₂HPO₄, NaCl, KCl, sodium ascorbate, sodium lactate, sodium gallate and combinations thereof.

19. The composition of claim 1 containing at least 95% water (by weight).

20. The composition of claim 19 containing at least 98% water (by weight).

21. The composition of claim 1 wherein the composition is reconstituted from a solid.

22. The composition of claim 21 wherein the solid is selected from a powder and a tablet.

23. The composition of claim 22 wherein the solid is a lyophilized powder.

24. The composition of claim 1 wherein the potency of the composition, as measured by agar diffusion assay, is at least equivalent to the antifungal activity of the equivalent amount of nystatin solution in DMF.

25. A water-based nystatin composition having improved bioavailability comprising:
nystatin,
2-4 eq of sodium deoxycholoate per eq of nystatin,
triethylamine,
a biological buffer,
water, and optionally
a salt
wherein the pH of the composition is in the range from about 7 to about 8.

26. The composition of claim 25 wherein the buffer is selected from the group consisting of Na₂HPO₄, TRIS, MOPS and combinations thereof.

27. The composition of claim 26 wherein the buffer comprises Na₂HPO₄.

28. The composition of claim 27 wherein the salt is selected from the group consisting of Na₂HPO₄, NaCl, KCl, sodium ascorbate, sodium lactate, sodium gallate and combinations thereof.

29. The composition of claim 25 wherein the potency of the composition, as measured by agar diffusion assay, is at least equivalent to the antifungal activity of the equivalent amount of nystatin solution in DMF.

30. A water-based antifungal composition having improved bioavailability comprising:
a polyene macrolide antifungal agent selected from the group consisting of amphotericin, candicidin, natamycin, polyfungin, Levorin and combinations thereof,
2-4 eq of a surfactant per eq of polyene macrolide antifungal agent, wherein the surfactant is selected from the group consisting of 3-[(3-cholamidopropyl)dimethylammonio]-1-propanesulfonate hydrate (CHAPS), sodium deoxycholate, sodium dodecyl sulfate (SDS), sodium myreth sulfate and combinations thereof;
an organic amine base selected from the group consisting of propylamine, ethanolamine, diethylamine, dipropylamine, trimethylamine, triethylamine, tripropylamine, diisopropylethylamine, diethyldimethylammonium hydroxide, tetrabutylammonium hydroxide, 4-methylmorpholine, morpholine and piperidine and combinations thereof,
a biological buffer selected from the group consisting of Na₂HPO₄, TRIS and MOPS,
water, and optionally
a salt selected from the group consisting of Na₂HPO₄, NaCl, KCl, sodium ascorbate, sodium lactate, sodium gallate and combinations thereof;
wherein the pH of the composition is in the range from about 7 to about 8.

31. A solid composition for preparing a water-based antifungal formulation comprising:
a polyene macrolide antifungal agent selected from the group consisting of nystatin, amphotericin, candicidin, natamycin, polyfungin, Levorin and combinations thereof,
a surfactant selected from the group consisting of 3-[(3-cholamidopropyl)dimethylammonio]-1-propanesulfonate hydrate (CHAPS), sodium deoxycholate, sodium dodecyl sulfate (SDS), sodium myreth sulfate and combinations thereof,
a biological buffer selected from the group consisting of Na₂HPO₄, TRIS, MOPS and combinations thereof, and
an organic amine base selected from the group consisting of propylamine, ethanolamine, diethylamine, dipropylamine, trimethylamine, triethylamine, tripropylamine, diisopropylethylamine, diethyldimethylammonium hydroxide, tetrabutylammonium hydroxide, 4-methylmorpholine, morpholine and piperidine and combinations thereof,
and optionally, a salt selected from the group consisting of Na₂HPO₄, NaCl, KCl, sodium ascorbate, sodium lactate, sodium gallate and combinations thereof.

32. The solid composition of claim 31 wherein the solid is selected from a powder and a tablet.

33. The solid composition of claim 32 wherein the solid is a lyophilized powder.
